(19) United States
(12) Patent Application Publication
(10) Pub. No.: US 2025/0178051 A1
(43) Pub. Date: Jun. 5, 2025

(54) METHODS OF CONTROLLING MICROBIOLOGICAL PROCESSES FOR IN SITU CONTAMINANT TREATMENT

(71) Applicant: Arizona Board of Regents on Behalf of Arizona State University, Scottsdale, AZ (US)

(72) Inventors: Justin SKINNER, Tempe, AZ (US); Anca DELGADO, Tempe, AZ (US); Nassar HAMDAN, Scottsdale, AZ (US); Jacob CHU, Phoenix, AZ (US)

(21) Appl. No.: 18/834,553

(22) PCT Filed: Jan. 31, 2023
(86) PCT No.: PCT/US23/61668

§ 371 Date: Jul. 30, 2024

Related U.S. Application Data
(60) Provisional application No. 63/305,239, filed on Jan. 31, 2022.

(51) Int. Cl. B09C 1/10; C12N 1/20

(52) U.S. Cl. CPC B09C 1/10; C12N 1/20; B09C 2101/00

(57) ABSTRACT

The present invention relates to a method for decreasing microbial biomass and biodegradation rates of in situ bioremediation system using acetylene as the inhibitor.

### CROSS-REFERENCE TO RELATED APPLICATIONS

This application claims the benefit of U.S. Provisional Application No. 63/305,239, entitled “Methods of Controlling Microbiological Processes For In Situ Contaminant Treatment,” which was filed Jan. 31, 2022, the entire disclosure of which is hereby incorporated herein by this reference.

### STATEMENT REGARDING FEDERALLY SPONSORED RESEARCH OR DEVELOPMENT

This invention was made with government support under 1449501 awarded by National Science Foundation. The government has certain rights in the invention.

### FIELD OF THE INVENTION

The invention relates to operational control of in situ aerobic bioremediation systems, including a system for co-metabolic trichloroethene (TCE) remediation.

### BACKGROUND OF THE INVENTION

In situ remediation of subsurface contaminant plumes via microbiological methods typically require the injection of microbial substrates (e.g., O₂, carbon and electron sources from organic compounds such as hydrocarbons, organic acids and alcohols, sources of N and P) and/or microbes that perform contaminant degradation. Injection of these substances increases the likelihood of rapid microbial growth near the injection site. A typical in situ microbial bioremediation system for groundwater contamination comprises an injection well and a monitoring well. The injection well has pumps to deliver desired augmentation materials (for example, O₂ or organic compounds) to the subsurface contamination zone. The monitoring well is a further a-field well that allow for monitoring of changes in the size and concentrations in groundwater contaminant plumes. Microbial growth can lead to bioclogging, as the soil pores are filled with microbial biomass and microbial products. The effects of bioclogging are especially pronounced in large diffuse groundwater plumes where contaminant concentrations are not present at sufficient levels to sustain metabolic growth thus necessitating the injection of microbial substrate. To prevent bioclogging, chemical substances that act as inhibitors to the microbes may be added as liquids or gases into the subsurface at the injection well(s). These inhibitors act to suppress or diminish microbial growth at the substrate injection points which further allows for enhanced operational control in the treatment zone.

Bioclogging reduces the permeability, porosity, and overall efficacy of subsurface treatment. In order to reduce the effect of bioclogging and enhance the efficacy of subsurface contaminant treatment, microbial inhibitors may be used to control biomass production rates. Hydrogen peroxide is an exemplary microbial inhibitor, but it is a non-specific oxidizing agent. Thus, novel microbial inhibitors and methods for applying them are needed for enhancing the effectiveness of biodegradation of contaminants.

### SUMMARY OF THE INVENTION

Described herein are methods of using acetylene as a microbial inhibitor for subsurface groundwater remediation, including removal of trichloroethene (TCE) contaminants. Using acetylene as a microbial inhibitor overcomes the limitations of other non-specific microbial inhibitors as acetylene is an enzyme-specific microbial inhibitor that feasibly allows for greater operational control. The methods prevent or minimizes the occurrence of bioclogging, which is a common problem across all in situ remediation regimes. The method employs a dose-dependent enzyme-specific microbial inhibitor which allows for operational control in the subsurface, thus conceivably enhancing biodegradation effectiveness. This increase in effectiveness further enhances the attractiveness of bioremediation technologies when compared to tradition pump and treat physical and chemical schemes.

In some embodiments, a method of controlling the rate of biomass production in an in situ bioremediation system is described. The method comprises administering acetylene to the in situ bioremediation system, wherein the in situ bioremediation system is exposed to acetylene for at least one day. In some aspects, the in situ bioremediation system is not exposed to treatment liquid for more than 10 days or for more than 8 days, for example, the in situ bioremediation system is exposed to acetylene for 1 day, 2 days, 4 days, or 8 days. The acetylene may be administered to the in situ bioremediation system by providing acetylene gas directly to the in situ bioremediation system or by providing acetylene-infused liquid (referred to hereinafter as “treatment liquid”), such as water bubbled with acetylene, to the in situ bioremediation system. In particular implementations, the treatment liquid is produced from bubbling acetylene through a liquid for at least four hours. In certain embodiments, the treatment liquid provided to the in situ bioremediation system at a rate of at least 0.2 mL/min for at least one three-hour pulses a day (such as two three-hour pulses in a day). Preferably, the treatment liquid comprises at least 1% v/v acetylene, at least 5% v/v acetylene, or about 5% v/v acetylene.

In some aspects, the in situ bioremediation system comprises a culture of bacteria that uses propane and oxygen as growth substrates, for example, Mycobacterium austroafricanum JOB5 or a mixed culture enriched from uncontaminated soil. In some implementations, the in situ bioremediation system degrades trichloroethene. In some aspects, the in situ bioremediation system removes subsurface contamination, for example the in situ bioremediation system is a soil column. In such embodiments, the in situ bioremediation system may comprise an injection site, and the acetylene is administered to the bioremediation system at the injection site.

Also described herein are a method of controlling the rate of biomass production in an in situ bioremediation system comprising providing a bacterial culture for in situ bioremediation; administering acetylene to the bacterial culture, wherein the bacterial culture is exposed to acetylene for at least one day to produce an inhibited bacterial culture; and providing the inhibited bacterial culture to the in situ bioremediation system. The administration of acetylene to the in situ bioremediation system inhibits microbial growth. In some aspects, acetylene exposure inhibits growth of microorganisms susceptible to bioclogging. Thus, a method of prohibiting bioclogging in an in situ bioremediation system comprising administering acetylene to the in situ bioremediation system is also described.

In yet another implementations, acetylene may be used to before bioaugmentation of an in situ bioremediation system to reduce endogenous bacterial diversity or during biostimulation or bioaugmentation to inhibit microbial growth to minimize the likelihood of bioclogging. Thus, methods of establishing an in situ bioremediation system comprising administering acetylene to a in situ bioremediation system before bioaugmentation, during biostimulation, or during bioaugmentation are also described.

### BRIEF DESCRIPTION OF THE DRAWINGS

FIGS. 1A-1E depict, according to certain embodiments, the changes in the concentration of TCE in cultures of Mycobacterium austroafricanum JOB5 or soil-derived propane-consuming mixed culture over eight days of acetylene exposure. The TCE consumption rates of the cultures of Mycobacterium austroafricanum JOB5 were 0.76 μmol TCE/(L day) on day 0 of acetylene exposure, 0.42 μmol TCE/(L day on day 1 of acetylene exposure, 0.63 μmol TCE/(L day) on day 2 of acetylene exposure, 0.36 μmol TCE/(L day) on day 4 of acetylene exposure, and 0.07 μmol TCE/(L day) on day 8 of acetylene exposure. The TCE consumption rates of the soil-derived propane-consuming mixed culture were 1.76 μmol TCE/(L day) on day 0 of acetylene exposure, 0.45 μmol TCE/(L day) on day 1 of acetylene exposure, 0.54 μmol TCE/(L day) on day 2 of acetylene exposure, 0.52 μmol TCE/(L day) on day 4 of acetylene exposure, and 0.25 μmol TCE/(L day) on day 8 of acetylene exposure.

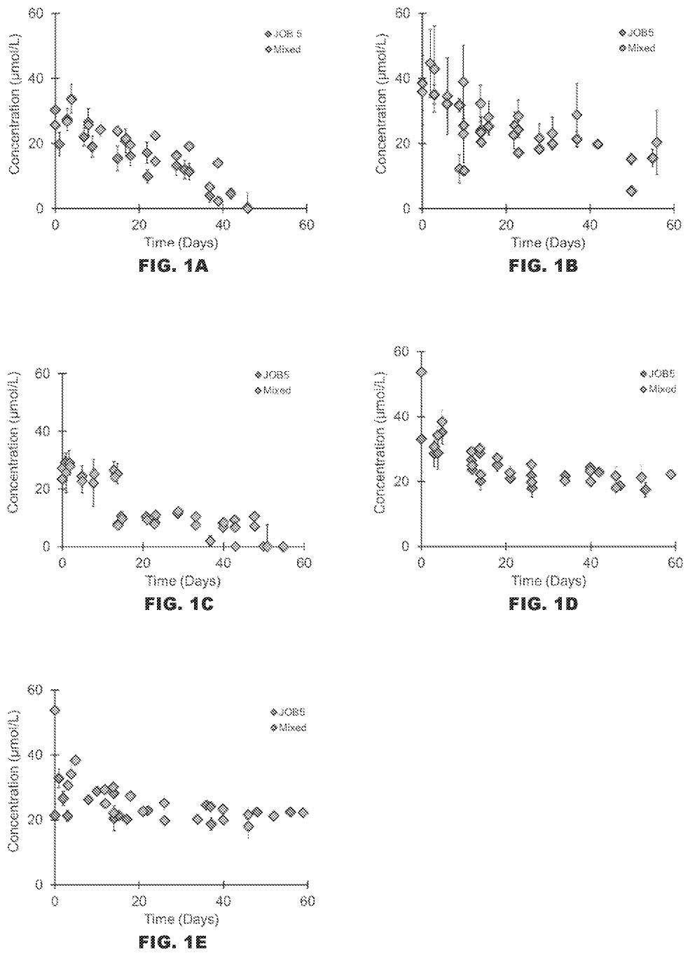

FIGS. 2A-2E depict, in accordance with certain embodiments, the changes in the concentration of optical density of cultures of Mycobacterium austroafricanum JOB5 or soil-derived propane-consuming mixed culture over eight days of acetylene exposure.

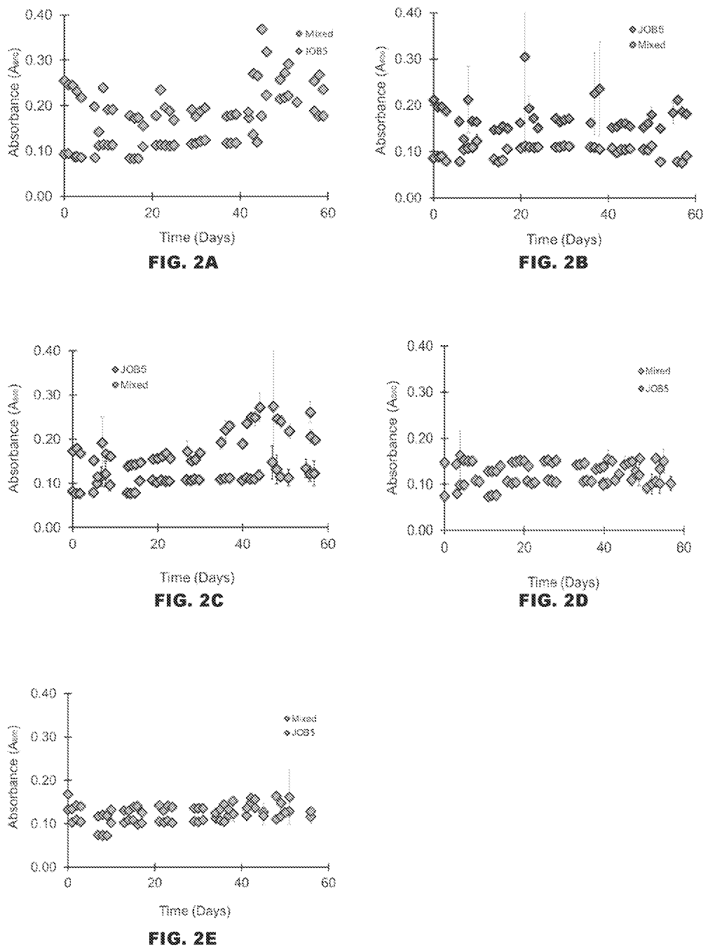

FIGS. 3A-3E depict, in accordance with certain embodiments, the partial pressures of the cultures of Mycobacterium austroafricanum JOB5 over eight days of acetylene exposure.

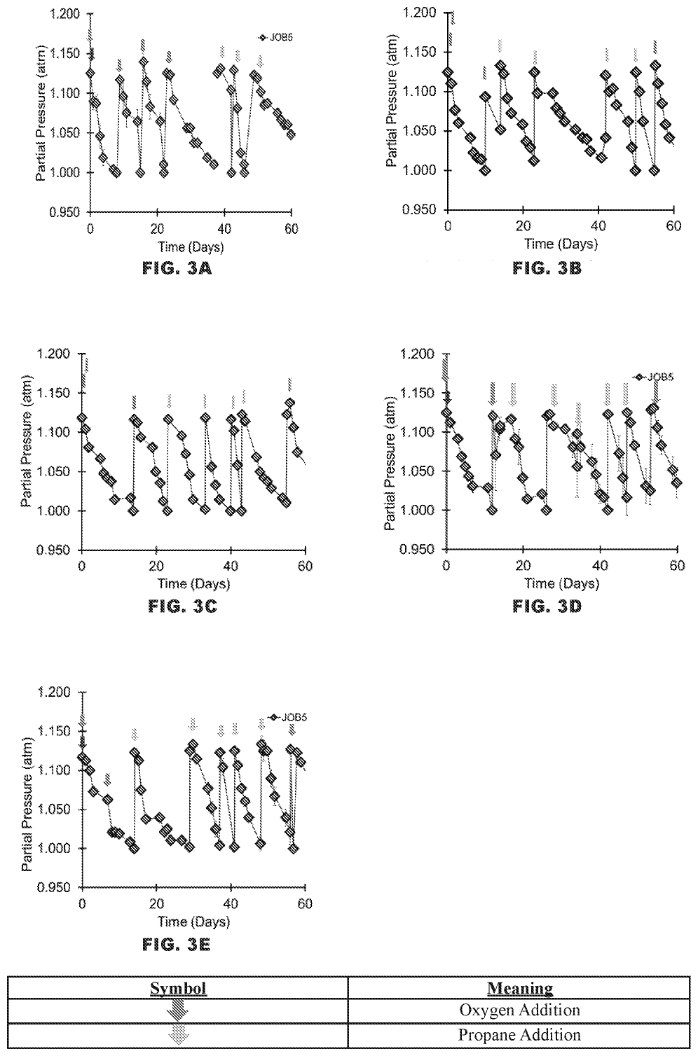

FIGS. 4A-4E depict, in accordance with certain embodiments, the partial pressures of the soil-derived propane-consuming mixed culture over eight days of acetylene exposure.

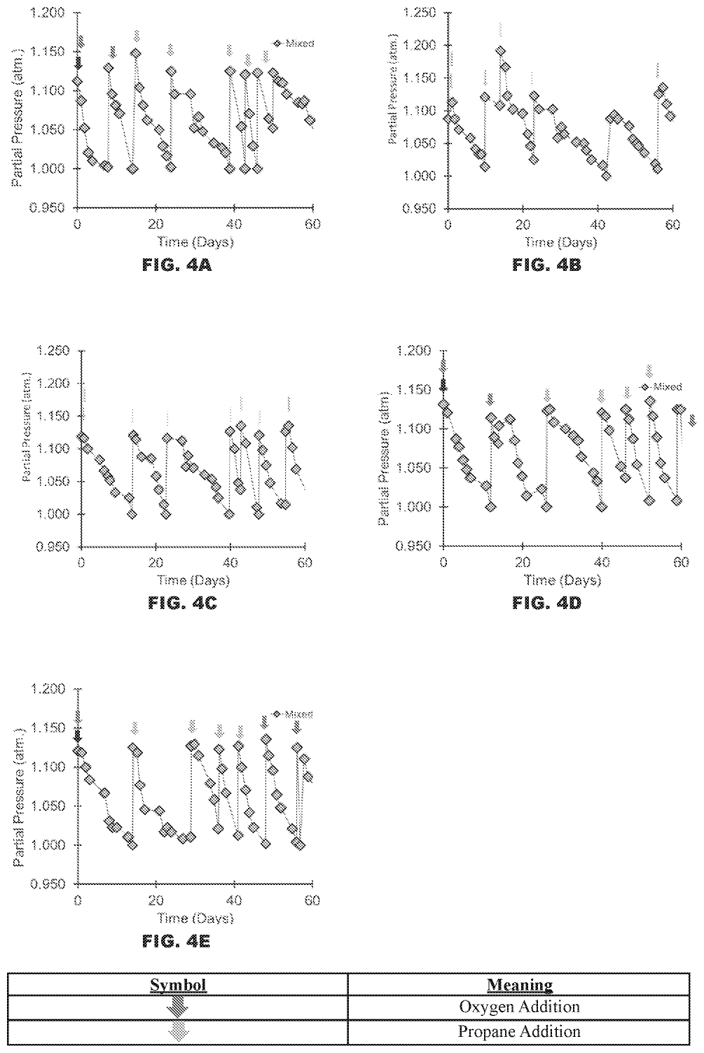

FIG. 5 depicts, in accordance with certain embodiments, the pumping schedule during flow through mode (Phase II).

FIGS. 6A-6F depict, in accordance with certain embodiments, the cumulative removal graphs of TCE removal (FIGS. 6A-6C) and propane consumption (FIGS. 6D-6F) dependent upon acetylene exposure in soil columns.

FIG. 7 depicts, in accordance with certain embodiments, acetylene's effect on biomass production in soil columns.

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “bioremediation” refers to the use of microbial metabolism in the presence of optimum environmental conditions and sufficient nutrients to breakdown contaminants.

As used herein, the term “biostimulation” refers to the step of adding limited nutrients to support microbial growth in a bioremediation system. In some aspects, the step involves modifying the environment in the bioremediation system to stimulate existing bacteria, such as providing a nutrient solution.

As used herein, the term “bioaugmentation” refers to the step of adding microorganisms capable of targeted microbial metabolism is the bioremediation system. Bioaugmentation may be involving adding native soil microorganisms or adding a non-native (foreign) population of microorganisms.

Disclosed herein is a novel method of controlling and enhancing in situ bioremediation processes based on the use of acetylene as a microbial inhibitor to minimize the occurrence of bioclogging. In situ bioremediation processes can be used to remediate subsurface contaminants, for example those in groundwater. The present disclosure is the first report of acetylene as a microbial inhibitor for subsurface groundwater remediation. In some aspects, the bioremediation processes include desulfurization, dehalogenation, denitrification, ammonification, and hydroxylation, of various aromatic and aliphatic contaminants. The bioremediation process may also include biotransformation and/or biodegradation of contaminants. In certain embodiments, the bioremediation processes involve cometabolism of contaminants. In particular implementations, the bioremediation processes involve the degradation of trichloroethene (TCE). For example, aerobic cometabolism of TCE is a viable methodology for TCE removal in large diffuse contaminant plumes. In the case of TCE cometabolism, an organic compound, such as a hydrocarbon (e.g., methane, propane, butane) is added as the growth-supporting substrate.

Acetylene is a suitable microbial inhibitor because it irreversibly bonds to the enzyme (monooxygenase) that is responsible for the metabolic degradation of simple hydrocarbons (for example, propane). Thus, unlike hydrogen peroxide, acetylene is an enzyme-specific microbial inhibitor. For certain implementations, acetylene also binds to the enzyme that is responsible for cometabolic degradation of TCE. The irreversible binding of acetylene to monooxygenases prevents the enzymes from binding with the metabolic substrates and prevents the organisms in the biomass from obtaining energy, which enables control of biomass growth. Acetylene inhibition may slowly be overcome as cells generate new monooxygenase enzymes, which also makes acetylene a suitable partial microbial growth inhibitor. An optimal concentration of acetylene as a microbial inhibitor would vary depending on the bioremediation system (for example, the depth of injection, depth of water table, porosity, groundwater flow velocity, and the dimensions of the bioremediation area). However, the optimal microbial inhibitory concentration would be a concentration that slows the microbial grown so that rapid growth does not occur near the injection site without negatively affecting the biodegradation of the bioremediation system.

As shown in the Examples, acetylene decreases microbial biomass and biodegradation rates. In certain implementations, administering 5% v/v (gaseous concentration) of acetylene to the in situ bioremediation resulted in an aqueous concentration of 2.2 mmol/L acetylene, which resulted in reduction of biomass demonstrating the ability this concentration of acetylene is sufficient to reduce and/or prevent bioclogging. The partial inhibition of microbial growth by acetylene in instances of aerobic TCE cometabolism may allow for greater overall contaminant treatment by reducing bioclogging near injection sites.

Acetylene may be suitable in any in situ processes where excess biomass is a challenge. The methods described herein is applicable to environmental engineering, geotechnical engineering, and other disciplines where bioclogging affects treatment efficacy. At a larger scale, the need for this invention arose from a common problem experienced in the in situ remediation of many contaminant types.

In certain implementations, the described method controls the rate of biomass production in an in situ bioremediation system. The method comprises providing a bioremediation system; and administering acetylene to the bioremediation system, wherein the bioremediation system is exposed to acetylene for at least one day. In some aspects, the bioremediation system comprises an injection site, the acetylene is administered to the bioremediation system at the injection site. The acetylene may be administered to the in situ bioremediation system by providing acetylene gas directly to the in situ bioremediation system or providing acetylene via a treatment liquid (such as water bubbled with acetylene) to the in situ bioremediation system. In particular implementations, the treatment liquid is produced from bubbling acetylene through a liquid (for example, groundwater). To ensure the treatment liquid contains a sufficient amount of acetylene, acetylene is preferably bubbled through the liquid for at least four hours. In certain embodiments, the treatment liquid provided to the in situ bioremediation system at a rate of at least 0.2 mL/min for at least one three-hour pulses a day (such as two three-hour pulses in a day). At least 1% v/v acetylene is administered to the bioremediation system, though in certain implementations, about 5% v/v acetylene or at least 5% v/v acetylene is administered to the bioremediation system. In some aspects, the bioremediation system comprises a culture of bacteria that uses propane and oxygen as growth substrates. For example, the culture of bacteria comprises Mycobacterium austroafricanum JOB5 or is a mixed culture enriched from uncontaminated soil. In certain embodiments, the bioremediation system degrades trichloroethene. In particular implementations, the bioremediation system removes subsurface contamination.

In certain implementations, the bioremediation system is not exposed to acetylene for more than 10 days. In other implementations, the bioremediation system is not exposed to acetylene for more than 8 days. In particular implementations, the bioremediation system is exposed to acetylene for 1 day, 2 days, 4 days, or 8 days.

Also described is a method of controlling the rate of biomass production in an in situ bioremediation system. In some aspects, the bioremediation system removes subsurface contamination. The method comprises providing a bacterial culture for in situ bioremediation; administering acetylene to the bacterial culture, wherein the bacterial culture is exposed to acetylene for at least one day to produce an inhibited bacterial culture; and providing the inhibited bacterial culture to the in situ bioremediation system. In some aspects, the bacterial culture is not exposed to acetylene for more than 10 days or for more than 8 days. In certain implementations, the bacterial culture is exposed to acetylene for 1 day, 2 days, 4 days, or 8 days. At least 1% v/v acetylene is administered to the bacterial culture, though in certain implementations, at least 5% v/v acetylene is administered to the bacterial culture. In particular implementations, 5% v/v acetylene is administered to the bacterial culture. In some embodiments, the culture of bacteria comprises Mycobacterium austroafricanum JOB or a mixed culture enriched from uncontaminated soil. In certain embodiments, the bacterial culture degrades trichloroethene. In some aspects, a method of prohibiting bioclogging in an in situ bioremediation system is disclosed. The method comprises administering acetylene to the in situ bioremediation system. In some aspects, the in situ bioremediation system is exposed to acetylene for at least one day to inhibit growth of microorganisms susceptible to bioclogging.

Methods of using acetylene to establish an in situ bioremediation system are also disclosed. In one aspects, the method comprises providing the in situ bioremediation system; and administering acetylene to the in situ bioremediation system prior to bioaugmentation, wherein the in situ bioremediation system is exposed to acetylene for at least one day to reduce endogenous bacterial diversity. In other aspects, the method comprises providing the in situ bioremediation system; and administering acetylene to the in situ bioremediation system during biostimulation or bioaugmentation, wherein the in situ bioremediation system is exposed to acetylene for at least one day to inhibit microbial growth.

### EXAMPLES

The present invention is further illustrated by the following examples that should not be construed as limiting. The contents of all references, patents, and published patent applications cited throughout this application, as well as the Figures, are incorporated herein by reference in their entirety for all purposes.

I. Acetylene Exposure Improves the Rate of Trichloroethene Degradation while Controlling the Rate of Biomass Production

Aerobic cometabolism of TCE is a viable methodology for TCE removal in large diffuse contaminant plumes. A pure culture of the known aerobic cometabolic TCE degrader, Mycobacterium austroafricanum JOB5 (formerly known as Mycobacterium vaccae JOB5) and a mixed culture enriched from an uncontaminated soil in mineral medium using propane and oxygen as growth substrates were used to test the efficiency of acetylene in reducing biomass growth rate (e.g., a proxy for bioclogging). The mixed culture was created through serial transfers from the original soil microcosms.

The pure and mixed cultures were then exposed to 50 μM TCE to verify their capacity to aerobically cometabolically degrade TCE. Once their capability to degrade TCE was verified, both cultures were grown in 2 L bottles and fed oxygen and propane until the optical density at 600 nm stabilized at ˜0.2 for multiple days. Then, the two cultures were exposed to acetylene at 5% v/v for varying lengths of time (no exposure (0), 1, 2, 4 and 8 days). The dissolved acetylene concentration in the 2 L bottles was 2.2 mmol/L.

After the set acetylene exposure time, culture was extracted, and triplicate bottles were started with 50 μM TCE and a pure oxygen headspace. The cultures were then monitored for propane (residual and added) and oxygen consumption, TCE degradation, carbon dioxide production, biomass production, and pH change.

Experiment data showed that both cultures were capable of aerobic TCE cometabolism and both cultures demonstrated exposure time-dependent relationships between TCE degradation rates, biomass production, and oxygen consumption rates (only results for no exposure, 4 days of exposure, and 8 days of exposure are shown). Specifically, non-inhibited treatments (no acetylene exposure) demonstrated the greatest TCE degradation and biomass production rates for both cultures tested (highest increase in optical density). As acetylene exposure times increased (1-8 days), the rates of TCE degradation and biomass production decreased. Under the conditions tested, acetylene exposure showed a time-dependent relationships on biodegradation rates and biomass production rates allowing it to be used as a microbial inhibitor for enhancing operational control of biodegradation reactions. High biomass is equated with increased likelihood of bioclogging. Thus, biomass production is a proxy for assessing bioclogging risk. As shown in the experimental data, there is less biomass production (optical density) in microcosms with longer exposure time to acetylene at 5% v/v. The partial inhibition of microbial growth by acetylene in instances of aerobic TCE

cometabolism allows for greater overall contaminant treatment by reducing bioclogging near injection sites. Increased effectiveness through prevention or reduction of bioclogging enhances the attractiveness of bioremediation processes over traditional pump and treat physical and chemical biodegradation schemes.

II. Acetylene-Containing Groundwater in Soil Columns with Contaminated Site Soil and Groundwater Decreased TCE Co-Metabolism Rates, Propane Consumption and Biomass Production

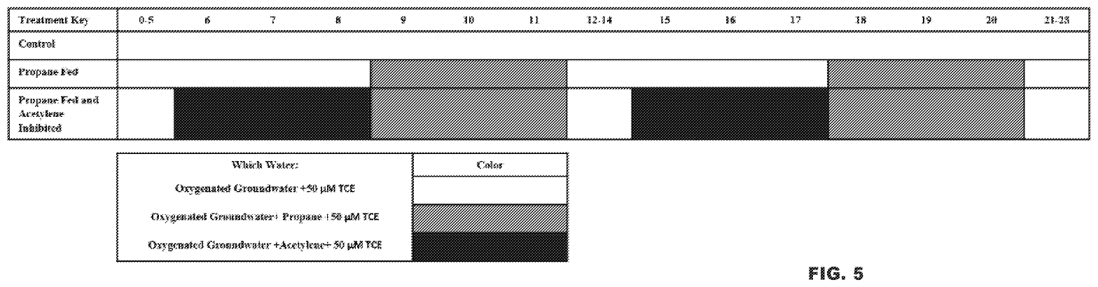

Groundwater was pumped at a 2-Day HRT (0.2 mL/min) from six pumps programmed to automatically turn on and turn off to deliver groundwater of varying chemistries to the soil columns. For the Propane-Fed (pumps 2-3) and the Propane-Fed and Acetylene-Inhibited treatments (pumps 4-6) which had multiple pumps feeding into one line PVC plastic check valves were installed at line splitting locations to avoid backflow to other feeder bags when pumps automatically started or stopped pumping according to the schedule presented in FIG. 5.

Acetylene or propane from a gas tank was bubbled into groundwater in a closed container in the lab. That groundwater was then pumped from closed container to a sealable Tedlar bag. The Tedlar bag was then connected to the pump which was controlled by a timer to deliver two three-hour pulses of acetylene containing groundwater at 0.2 mL/min per day. The pump effectively serves as a seal for the Tedlar bag as the tubing is pinched at one location to create suction.

For propane, 0.5 L of propane was bubbled through 10 L water for at least four hours. That propane-containing water was then pumped into a Tedlar bag. For the treatments which received propane-containing water, the water was pumped into the columns at 0.2 mL/min to 6 hrs/day (two three-hour pulses). For acetylene, 0.5 L of acetylene was bubbled through 10 L water for at least four hours. That acetylene-containing water was then pumped into a Tedlar bag. For the treatments which received acetylene-containing water, the water was pumped into the columns at 0.2 mL/min to 6 hrs/day (two three-hour pulses)

Soil columns were bioaugmented with a mixture of Mycobacterium austroafricanum JOB-5 (JOB-5) and a mixed culture, named COMET1, enriched from a soil at Arizona State University (Tempe, Arizona, United States).

TABLE 1
Groundwater Pumping Key

| | | | | Volume per day |
|---|---|---|---|---|
| | Pumping to | | Hours pumping | per column |
| Pump # | Which Treatment | Delivering which water | per day | (mL) |
| 1 | Control | Oxygenated GW + 50 | 24 | 288 |
| | | μM TCE | | |
| 2 | Propane Fed | Oxygenated GW + 50 | 18 | 216 |
| | | μM TCE | | |
| 3 | Propane Fed | Oxygenated GW + | 6 | 72 |
| | | Propane + 50 μM TCE | | |
| 4 | Propane Fed and | Oxygenated GW + 50 | 12 | 144 |
| | Acetylene | μM TCE | | |
| | Inhibited | | | |
| 5 | Propane Fed and | Oxygenated GW + | 6 | 72 |
| | Acetylene | Propane + 50 μM TCE | | |
| | Inhibited | | | |
| 6 | Propane Fed and | Oxygenated GW + | 6 | 72 |
| | Acetylene | Acetylene + 50 μM | | |
| | Inhibited | TCE | | |

Phase I involves bioaugmentation in soil columns in batch mode. From the COMET1 and JOB5 mother cultures as described above, 450 mL of each culture was removed and placed in a priorly autoclaved and sealed beaker. This 900 mL mixture of the COMET1 and JOB5 cultures was mixed on a stir plate and then 50 mLs of the mixture was injected into each groundwater sampling port (3 ports×6 columns). After culture injection 1 HRT of Oxygenated Groundwater+Propane was flowed through all columns except the control treatment. After 1 HRT, 10 mL of nutrient solution (comprising, in 1 L, 10 g sodium chloride, 5 g magnesium chloride hexahydrate, 20 g potassium phosphate monobasic, 30 g ammonium chloride, 30 g potassium chloride, and 0.5 g calcium chloride dihydrate) was added to each column. The columns were sealed with water samples extracted every two days. Batches were continued until either propane was not detected in the groundwater or propane consumption slowed. Thereafter, columns were unsealed and flowed through again via the above-described procedure. This biomass augmentation procedure was repeated 2 times prior to experimentation.

Phase II involves flow through mode. After the batch mode bioaugmentation phase, columns were unsealed and flowed at 0.2 mL/min from the feeder bags and pumping schedule as described above. Daily checks occurred to ensure that pumps were functioning according to desired flow rates and pumping schedule. Columns were sampled roughly every two days during the experiment when instrumentation was available. Gas sampling consisted of taking 2×0.5 mL from both the pre- and post-column flow through gas vials. These gas syringes were then analyzed for TCE, propane, and acetylene via gas chromatography with flame ionization detection (GC-FID) and for oxygen and carbon dioxide via assessment of a thermal conductivity detector (TCD). Details of the analysis methods are described in Materials and Methods. One mL of water was sampled during each sampling event from the bottom and top sampling ports and frozen for later analysis.

a. Materials and Methods

Chemicals: Propane (99%+), acetylene (99%+), and ultra-high purity oxygen (O₂) (99.999%) were obtained from Matheson Tri-Gas Inc. (Irving, TX). TCE (≥99.5%) was obtained from Aldrich Chemical Company (Milwaukee, WI).

Chemical analytical methods: TCE, propane, and acetylene were measured by injecting 200 μL gas samples into a gas chromatograph (GC, Shimadzu GC-2010; Columbia, MD) equipped with a flame ionization detector and a Rt-QS-BOND capillary column (Restek; Bellefont, PA). The instrument settings and method were described elsewhere (Delgado, Parameswaran et al. 2012, Rangan, Mouti et al. 2020, Joshi, Robles et al. 2021, Robles, Yellowman et al. 2021). The calibration ranges were as follows: TCE, 1-220 μM; propane, 0.5-13 mM; and acetylene, 0.5-10 mM. The minimum detection limits were 0.2 μM for TCE, 0.02 μM for propane, and 0.15 mM for acetylene.

O₂ and CO₂ concentrations were determined by injecting 200 μL gas samples into a GC (Shimadzu GC-2010) equipped with a thermal conductivity detector (TCD) and a fused silica capillary column (Carboxen1010 PLOT, Supelco, Bellefonte, PA) (Joshi, Robles et al. 2021). The carrier gas was Ar, the injector temperature was 150° C., and the detector was set at 41 mV. The calibration ranges were as follow: 0.3-10.5 mmol L⁻¹ O₂ (gas concentration) and 0.05-5 mmol L⁻¹ CO₂ (gas concentration). The minimum detection limits for O₂ and CO₂ were 0.2 mmol L⁻¹ and 0.13 mmol L⁻¹ (gas concentrations), respectively.

Liquid samples (1 mL) were used to measure pH using a Sartorius pH bench top meter (Thermo Scientific, Waltham, MA) equipped with an Orion economy series pH electrode and calibrated with Orion pH 4.01, 7.00, and 10.01 standard solutions.

Total Organic Carbon Measurement Procedure: Total Organic Carbon (TOC) was assessed as a proxy measurement for biomass production in the soil columns using a Shimadzu Solid Sample Module (SSM) 5000A Carbon Analyzer (Tokyo, Japan). TOC analysis was performed both prior to and post-experimentation. To sample sediment from the columns post-experimentation, we sacrificially sampled a column by cutting a segment of the column lengthwise and removed sediment samples at differing lengths along the column. Sediment sampling for DNA extraction at various lengths along the column was carried out via the same method. To prepare soil samples for TOC analysis, soil samples were dried in a Fischer Scientific (Waltham, MA) Isotemp Oven at 105° C. for four hours. After drying, samples were placed in a desiccator for one hour. Samples were then ground using a ceramic mortar and pestle and sieved with a Soiltest Incorporated 200 mesh sieve (0.074 mm) as instructed by the Shimadzu TOC-SSM 5000A protocol. Between 240-260 mg of a ground sample, with specific weight of each sample recorded, was loaded onto a ceramic TOC-SSM measuring boat. Between sampling and before reuse, the measurement boats were DI rinsed, acid washed for 10 min in 2M sulfuric acid, DI rinsed again and baked in aluminum foil in a Barnstead Thermolyne (Ramsey, MN) 30400 Furnace at 550° C. for 20 min to remove all carbon. Ceramic measuring boats were only handled using flame-sterilized tweezers and placed on aluminum foil during measurement preparation to avoid carbon contamination A clean empty boat was measured prior to each sample batch to ensure that boats were carbon free prior to soil addition.

Ceramic boats were then loaded into the TOC-SSM module for TOC measurement. Samples were measured in triplicate. A calibration curve was generated using alpha-D (+)-Glucose, 99+%, anhydrous range from 9-158 μg carbon (R2-0.9993) with a detection limit of 3 μg carbon. After the samples were loaded onto the boats the sediment samples were cover with ceramic fiber to avoid sediment splattering during measurement. Samples were then loaded into the sampling port and combusted at 980° C. as per Shimadzu's standard SSN TOC method. Generated peaks were automatically integrated and conversion to % TOC occurred via the TOC-Control L software.

Calculations: The aqueous and gas concentrations of TCE, propane, acetylene, and CO₂ in the flow through gas measurement vessels were converted to nominal concentrations in units of umol L⁻¹ or mmol L⁻¹ using dimensionless Henry's law constants (H^{CC}) according to Equation 1:

$${C}_{n} = \frac{( {V}_{g} \times {C}_{g} ) + ( {V}_{1} \times \frac{{C}_{g}}{{H}^{CC}} )}{{V}_{1}} \qquad Equation 1$$

where V_{g} is the headspace volume (L), C_{g} is the gas concentration in the headspace (umol L⁻¹ or mmol L⁻¹), V₁ is the liquid volume (L), and H^{CC} is the dimensionless Henry's law constant for the compound. H^{CC} was calculated according to Equation 2:

$${H}^{CC} = {H}^{CP} \times RT \qquad Equation 2$$

where H^{CP} is the dimensional Henry's law constant (mol/m³ Pa), R is the ideal gas constant

$$( 8.314 \frac{{m}^{3} Pa}{Kmol} )$$

and T is the temperature (K). Values for H^{CP} were obtained from these sources (Warneck 2007, Warneck 2012, Sander 2015). The dimensionless H^{CC} used were as follows: TCE, 0.3; acetylene, 0.98; propane, 26.91; O₂, 31.25; CO₂, 1.22.

Cumulative removal graphs were generated via the following procedure. The concentration of the analyte in the effluent was subtracted from the concentration in the influent. This was then multiplied by the pore volume as verified from the previously described porosity determination and then divided by the mass of soil in the column. This data operation is described in Equation 3 below. These column-specific data were then cumulatively summed and then averaged to generate treatment-specific graphs.

$${\sum }_{Start of Experiment}^{n} \frac{( Influent - Effluent ) * Pore Volume}{Mass of Soil} = \frac{μM or mM}{g soil} \qquad Equation 3$$

Conversion of TOC peak area to μg carbon occurred via the generated calibration curve described above and μg carbon was converted to percent TOC via Equation 4 below.

$$TOC ( \% ) = \frac{Mass of Carbon in Sample ( mg )}{Mass of Soil ( mg )} * 100 \qquad Equation 4$$

b. Results and Discussion

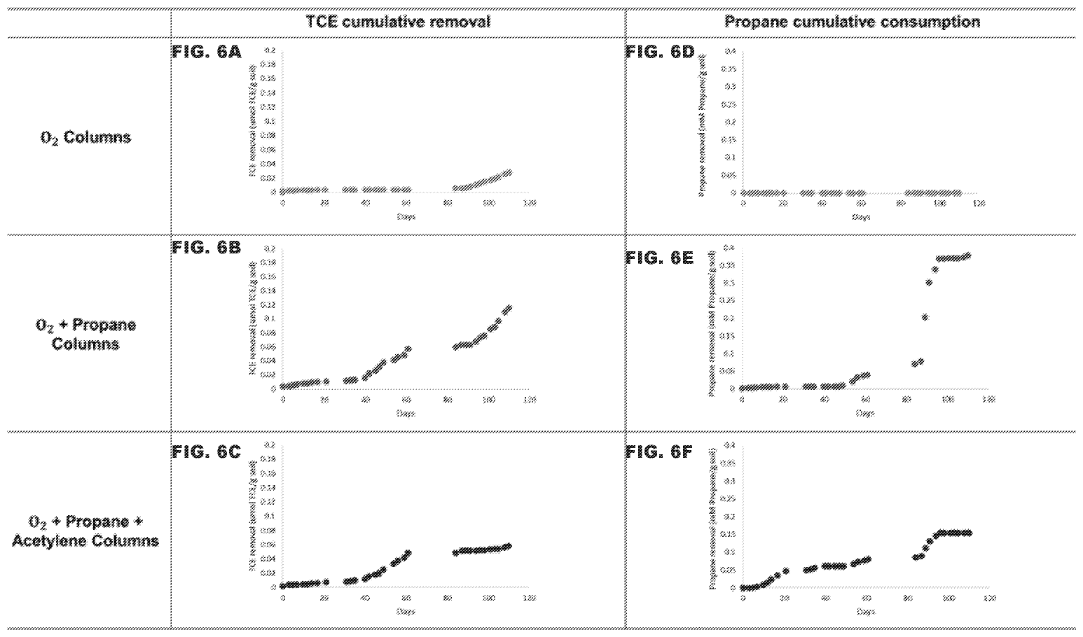

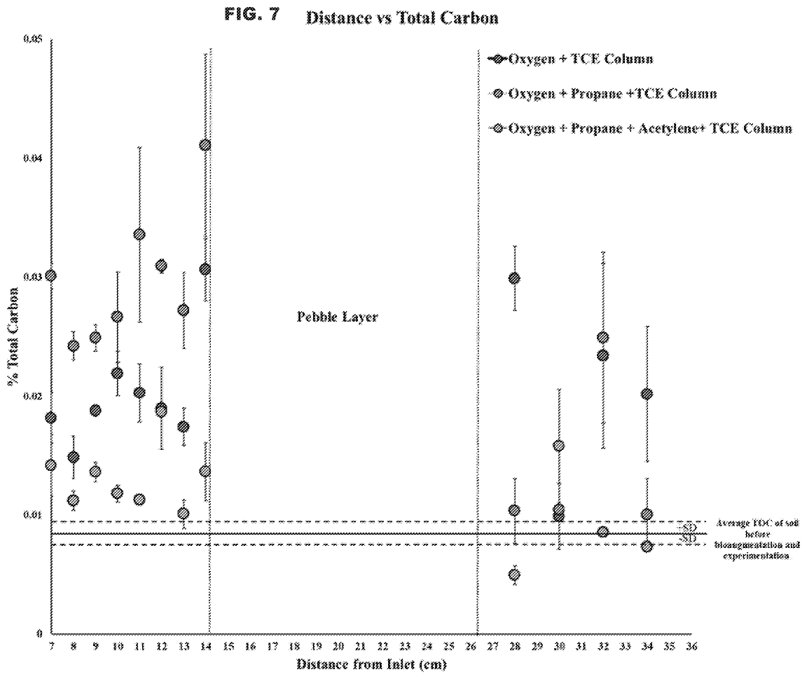

The experiment confirmed that relationships exist between acetylene exposure and cumulative rates of TCE co-metabolism rates, propane consumption, and biomass production (FIGS. 6A-6F and 7). Specifically, acetylene exposure slowed TCE degradation, propane consumption, and biomass production.

As shown in FIGS. 6A-6F, acetylene inhibits aerobic TCE co-metabolism, slow co-metabolizing communities' propane consumption rates. FIG. 7 shows acetylene exposure is associated with lower biomass presence in soil columns containing contaminated site sediment and groundwater. Therefore, acetylene functions as a microbial inhibitor that aids enhancing growth rate control of contaminant degrading microbial communities, which are frequently associated with well and subsurface overgrowth that results in bioclogging. Acetylene is a remedy for a common in situ bioremediation challenge that if overcome will allow in situ bioremediation to perform more reliably and cost-effectively.

1. A method of controlling the rate of biomass production in an in situ bioremediation system, the method comprising administering acetylene to the in situ bioremediation system, wherein the in situ bioremediation system is exposed to acetylene for at least one day.

2. The method of claim 1, wherein acetylene is administered to the in situ bioremediation system through a treatment liquid, wherein the treatment liquid is produced from bubbling acetylene through a liquid.

3. The method of claim 3, wherein the treatment liquid is produced from bubbling acetylene through a liquid for at least four hours.

4. The method of claim 2, wherein the treatment liquid provided to the in situ bioremediation system at a rate of at least 0.2 mL/min for at least one three-hour pulses a day.

5. (canceled)

6. (canceled)

7. The method of claim 1, wherein the in situ bioremediation system is exposed to acetylene for 1 day, 2 days, 4 days, or 8 days.

8. The method of claim 2, wherein the treatment liquid comprises at least 1% v/v acetylene, at least 5% v/v acetylene, or about 5% v/v acetylene.

9. The method of claim 1, wherein acetylene gas is delivered to the in situ bioremediation system.

10. The method of claim 1, wherein the in situ bioremediation system comprises a culture of bacteria that uses propane and oxygen as growth substrates.

11. The method of claim 10, wherein the culture of bacteria comprises Mycobacterium austroafricanum JOB5.

12. The method of claim 10, wherein the culture of bacteria is a mixed culture enriched from uncontaminated soil.

13. The method of claim 10, wherein the in situ bioremediation system degrades trichloroethene.

14. The method of claim 1, wherein the in situ bioremediation system removes subsurface contamination.

15. (canceled)

16. The method of claim 1, wherein the in situ bioremediation system comprises an injection site, the acetylene is administered to the bioremediation system at the injection site.

17. A method of controlling the rate of biomass production in an in situ bioremediation system, the method comprising:
providing a bacterial culture for in situ bioremediation;
administering acetylene to the bacterial culture, wherein the bacterial culture is exposed to acetylene for at least one day to produce an inhibited bacterial culture; and
providing the inhibited bacterial culture to the in situ bioremediation system.

18. The method of claim 17, wherein the culture of bacteria bacterial culture comprises Mycobacterium austroafricanum JOB, or a mixed culture enriched from uncontaminated soil.

19. The method of claim 17, wherein the bacterial culture degrades trichloroethene.

20. (canceled)

21. The method of claim 17, wherein the bacterial culture is not exposed to acetylene for more than 10 days or for more than 8 days.

22. (canceled)

23. The method of claim 17, wherein at least 1% v/v acetylene, at least 5% v/v acetylene, or about 5% v/v acetylene is administered to the bacterial culture.

24. (canceled)

25. (canceled)

26. A method of establishing an in situ bioremediation system, the method comprising:
providing the in situ bioremediation system; and
administering acetylene to the in situ bioremediation system prior to bioaugmentation, wherein the in situ bioremediation system is exposed to acetylene for at least one day to reduce endogenous bacterial diversity.

27. (canceled)

28. The method of claim 26, wherein the in situ bioremediation system is not exposed to treatment liquid for more than 10 days or for more than 8 days.
